(19) United States
(12) Patent Application Publication
(10) Pub. No.: US 2024/0215642 A1
(43) Pub. Date: Jul. 4, 2024

(54) Liquid Jet Inhalation Device

(71) Applicant: JT International SA, Geneva (CH)

(72) Inventors: James Alan Mott, San Diego, CA (US); Simon Dodd, Albany, OR (US); Gianluca Gulli, Sesto San Giovanni (IT); Domenico Giusti, Caponago (IT); Bruce Michael Eckard, San Diego, CA (US); Jeffrey John Glassett, Escondido, CA (US); Joseph Winn Fiske, San Marcos, CA (US)

(73) Assignee: JT International SA, Geneva (CH)

(21) Appl. No.: 18/288,710

(22) PCT Filed: Apr. 22, 2022
(86) PCT No.: PCT/EP2022/060709

§ 371 Date: Oct. 27, 2023

(30) Foreign Application Priority Data
May 6, 2021 (EP) 21172471.1

(51) Int. Cl. A24F 40/46; A24F 40/485; A61M 11/02; A61M 11/04

(52) U.S. Cl. CPC A24F 40/46; A24F 40/485; A61M 11/02; A61M 11/042

(57) ABSTRACT

An inhalation device with at least one liquid jet device for producing drops of a liquid on demand, wherein the liquid jet device includes a fluid chamber, an ejection nozzle, a supply channel embedded in a substrate, and at least one heating element arranged to pre-heat the liquid to a predetermined temperature prior to ejection through the ejection nozzle.

### TECHNICAL FIELD

The present invention relates to inhalation devices with at least one liquid jet device for producing drops of a liquid on demand. More particularly, the present invention relates to such inhalation devices in the form of electronic cigarettes, cigalikes, e-cigarettes, vapour inhalers and related devices.

Generally, with regard to any further details, the disclosure of the applicant's applications entitled “INHALATION DEVICE WITH AT LEAST ONE LIQUID JET DEVICE, FOR CARTRIDGE AN INHALATION DEVICE AND METHOD OF CONVEYING LIQUID DROPS IN AN INHALATION DEVICE”, “CONTAINER FOR AN INHALATION DEVICE WITH AT LEAST ONE LIQUID JET DEVICE, COMBINATION OF AT LEAST TWO CONTAINERS AND METHOD OF CONVEYING LIQUID TO AN INHALATION DEVICE”, and “LIQUID JET INHALATION DEVICE” filed on the same day are incorporated herein by means of the reference.

### BACKGROUND

In the arts there are several types and concepts for inhalation devices that serve a broad range of purposes including medical and therapeutic applications and also leisure and pleasure devices such as electronic cigarettes. Existing inhalation devices either change the phase of a fluid before inhalation with for example a wick and a coil so as to significantly raise the vapor temperature above human body temperature or deliver drops a room temperature by, for example, employing an ultrasonic mesh. In the above wick and coil system the vapor can be perceived as ‘warm’ by a user in the mouth, whereas in the ultrasonic mesh systems, the vapor is usually perceived as ‘cold’.

Such inhalation devices are oftentimes portable and pocket-size devices that can easily fit in the user's hand or can be handled by the user's fingers alone. In this way, the devices can be convenient for use and can be carried by a user for regular or emergency use. In the former case, a user can conveniently carry an inhalation device in the form of an e-cigarette to use and enjoy it whenever and wherever desired, whereas in the latter case a medical or therapeutic inhalation device may be ready to use when needed.

As such devices generate from a liquid drops or vapour that is to be inhaled by a user, the temperature of the inhaled aerosol/air mixture is can be of substantial importance: First, the absorption by the body of any active medical agents may highly depend on temperature. Second, also in the case of aromas and leisure applications, the temperature of the inhaled mixture may greatly affect user experience. In addition to these more or less objective reasons, the temperature of the inhaled mixture may of course also be subject to individual taste and preferences.

There is therefore a need for improved inhalation devices that not only ensure convenience in use and carriage, but also improve quality of experience and fidelity of action. It is thus an object of the present invention to provide such improved inhalation devices that can remedy the drawbacks of the conventional solutions.

### SUMMARY

The mentioned drawbacks are remedies by the subject-matter of the independent claims. Further preferred embodiments of the present invention are defined in the dependent claims.

According to one embodiment of the present invention there is provided an inhalation device with at least one liquid jet device for producing drops of a liquid on demand, said liquid jet device comprising a fluid chamber, an ejection nozzle and a supply channel embedded in a substrate, the inhalation device further comprising at least one heating element arranged to pre-heat said liquid to a predetermined temperature prior to ejection through said ejection nozzle.

### BRIEF DESCRIPTION OF THE DRAWINGS

Embodiments of the present invention, which are presented for better understanding the inventive concepts and which are not to be seen as limiting the invention, will now be described with reference to the Figures in which:

FIGS. 1A to 1C show schematic views of an inhalation device according to respective embodiments of the present invention;

FIG. 2A shows a schematic view of a MEMS configuration in a substrate according to a respective embodiment of the present invention;

FIG. 2B shows a schematic view of a MEMS layout on a substrate according to a respective embodiment of the present invention;

and

FIGS. 3A and 3B show schematic views of vapour generator assemblies of an inhalation device according to respective embodiments of the present invention.

### DETAILED DESCRIPTION

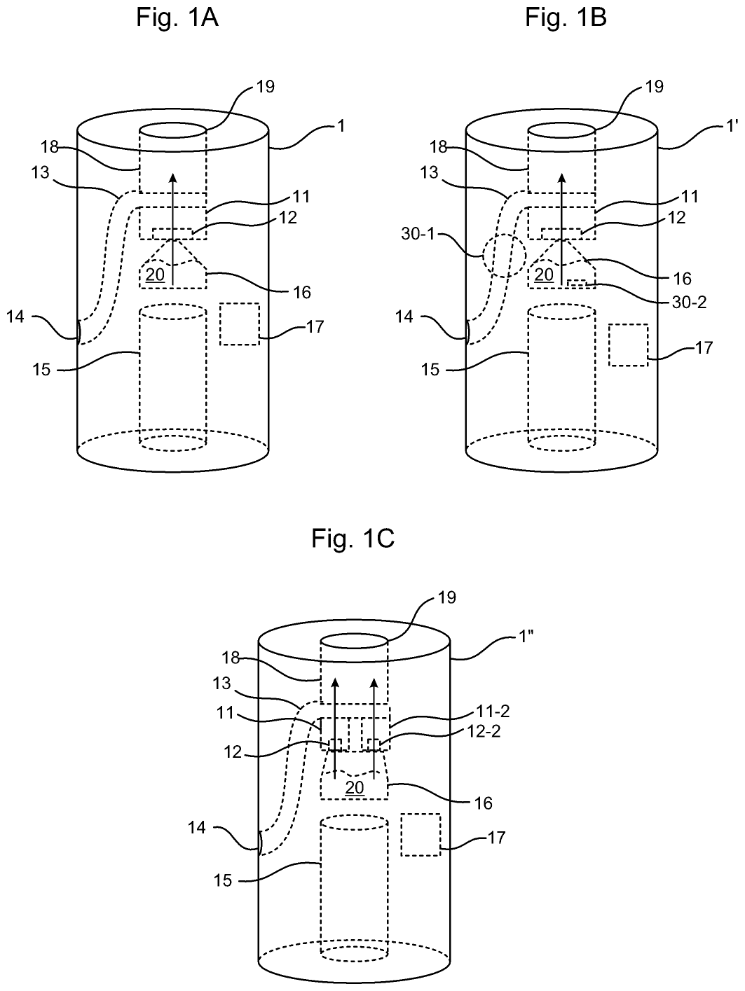

FIGS. 1A to 1C show schematic views of an inhalation device according to respective embodiments of the present invention. In FIG. 1A there is shown an inhalation device 1 that comprises at least one liquid jet device 11 for producing drops of a liquid on demand, which, in turn, comprises a fluid chamber, an ejection nozzle and a supply channel embedded in a substrate. Such details of the liquid jet device are explained and describer in greater detail elsewhere in the present disclosure. The inhalation device 1 further comprises at least one heating element 12 arranged to pre-heat a liquid 20 to a predetermined prior to ejection through said ejection nozzle.

The inhalation device 1 further comprises an air conduit 13 and a mixing or mouthpiece chamber 18 in which air from said air conduit 13 is mixed with the liquid drops generated by the liquid jet device 11. The air conduit 13 further comprises at least one air inlet orifice 14 at some suitable site of said inhalation device 1. The inhalation device 1 may further comprise a reservoir 16 for storing an amount of said liquid 20 to be vaporized, a power source 15 in the exemplary form of a battery or a rechargeable battery, and a controller 17 that is configured to control all necessary parts and functions of the inhalation device 1. Liquid reservoirs, power sources and controllers (apart from the specific control program employed in the embodiments of the present invention) are as such available from the conventional arts, so that greater details of these elements are omitted here. Preferably, the inhalation device may comprise a reservoir configured to store an amount of said liquid and a reservoir heating element arranged to heat the liquid in said reservoir to a predetermined liquid reservoir temperature.

The inhalation device 1 can further comprise a mouthpiece chamber to which a mouthpiece can be connected so a user can conveniently inhale the generated aerosol/vapour/air mixture. Specifically, the air conduit 13 and the mouthpiece chamber 19 are provided so that air from said air conduit 13 can be mixed with the generated liquid drops. The inhalation device 1 can further comprise a reservoir 16 configured to store an amount of said liquid 20.

FIG. 1B shows a schematic view of an inhalation device 1′ according to a further embodiment of the present invention. The device 1′ comprises similar, identical or equivalent elements as the inhalation device 1 described in conjunction with FIG. 1A. However, this embodiment considers the option that the inhalation device 1′ has an air conduit 13 with an optional further heating element 30-1 arranged to pre-heat the air guided by the air conduit 13 into the mixing or mouthpiece chamber 18. As an alternative thereto or in addition to this, the inhalation device 1′ further comprises a reservoir 16 configured to store an amount of said liquid with a reservoir heating element 30-2 arranged to heat the liquid in said reservoir to a predetermined liquid reservoir temperature. In this way additional pre-heating of the liquid 20 can be obtained by the heating element 30-1 and/or the heating element 30-2.

FIG. 1C shows a schematic view of an inhalation device 1″ according to a further embodiment of the present invention. The device 1″ comprises similar, identical or equivalent elements as the inhalation device 1 and l′ described in conjunction with FIG. 1A and/or 1B. In this embodiment, the inhalation device 1″ comprises at least one further liquid jet device 11-2 for producing drops of a liquid on demand, said further liquid jet device comprising again a fluid chamber, an ejection nozzle and a supply channel embedded in a further substrate (described elsewhere).

The inhalation device 1″ can comprise at least one further heating element 12-2 arranged to pre-heat a liquid to a further predetermined temperature prior to ejection through the ejection nozzle further in the substrate. In one embodiment, the liquid jet device 11 and said further liquid jet device 11-2 are provided for producing drops from respective different liquids, whereas in an alternative, the liquid jet device 11 and said further liquid jet device 11-2 are provided for producing drops from the same liquid 20. It should be clear that the embodiments of FIGS. 1A and 1C can be combined with that of FIG. 1B, so that additional pre-heating of the liquid (s) can be provided for inhalation devices that provide one or more than one liquid jet device.

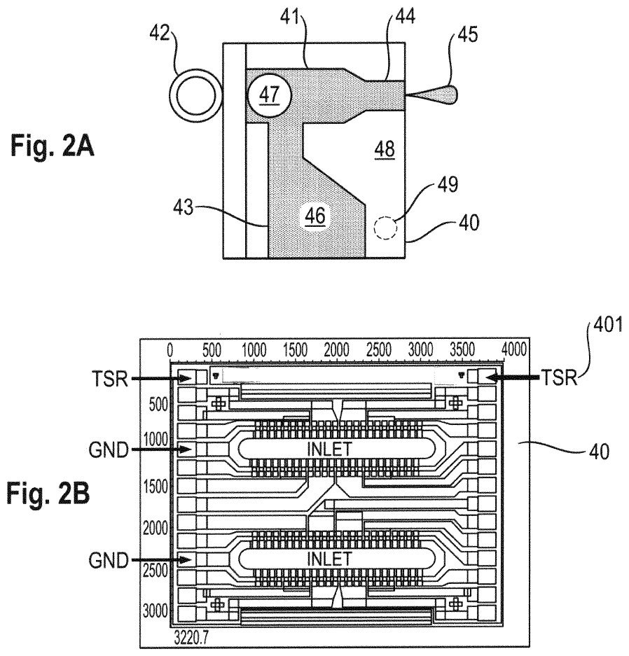

FIG. 2A shows a schematic view of a MEMS configuration in a substrate according to a respective embodiment of the present invention. The liquid jet device 40 is formed as a MEMS in a substrate 48 of any suitable material, for example silicon. In that substrate 48 there is formed a fluid chamber 41, an ejection nozzle 44, and a supply channel 43 in liquid communication with a reservoir for providing liquid 46 to the fluid chamber 41 so as to be vaporized or atomized. An ejection heating element 42 is arranged in the vicinity of the fluid chamber 41 so as to heat up a portion of the liquid 46 to vaporized and form a gas bubble 47. Specifically, said ejection heating element 42 can be a resistor arranged in a vicinity of said fluid chamber 41 and configured to heat a an amount of the liquid 46 to at least a vaporization temperature, so that a vapour bubble expels a drop of the liquid through the ejection nozzle. So, the resulting expansion leads to the ejection of an amount of the liquid in the form of a drop or droplet 45 that then can form in the mixing chamber a vapour or aerosol.

In the embodiment, there is provided a heating element 49 arranged to pre-heat said liquid 46 to a predetermined temperature prior to ejection through said ejection nozzle 44. For example, the heating element 49 can be a resistor embedded in the substrate 48 so as to pre-heat to the predetermined temperature at least a part of the liquid 46 present in the fluid chamber 41 or supply channel 43. In this way, the resistor embedded in a substrate of said MEMS can pre-heat to a predetermined temperature at least a part of the liquid present in cavities formed in the substrate 48. The overall control (e.g. control unit 17) may be configured to drive said resistor 49 so that an amount of the liquid 46 is pre-heated before entering the fluid chamber 41 to said predetermined temperature, and then subsequently the amount of the liquid is heated to at least the vaporization temperature. In this way, the temperature of the liquid can be raised just before being ejected. Generally, said resistors can be a temperature sense resistor, TSR, embedded in the substrate.

An embodiment, in which a resistor is a temperature sense resistor 401, TSR, embedded in the substrate 40 is shown in FIG. 2B. Here, the TSR 401 is used for heating, in which a MEMS die is provided with a TSR trace to measure the temperature of the substrate (e.g. silicon) and, in steady state, the temperature of the liquid being ejected. This can be a trace with high current carrying capability. By putting voltage to the TSR, heat can be generated through resistance.

In a further embodiment the heating element can be realized by the heating resistor 42 arranged in a vicinity of the fluid chamber 41 and configured to heat an amount of the liquid in the fluid chamber to at least the vaporization temperature, so that the vapour bubble 47 expels a drop 45 of the liquid through the ejection nozzle 44. In such an embodiment, the control unit can be configured to drive said resistor 42 so that the amount of the liquid in the firing chamber 41 is first pre-heated to the predetermined temperature prior to heating the amount of the liquid to at least the vaporization temperature. Preferably, the control unit can be configured to apply a voltage pulse with variable width to said resistor 42, and a pre-heating pulse is shorter than a vaporization pulse. Even more preferably, the pre-heating pulse is approximately half as long as said vaporization pulse.

In general, temperatures above 150° C. should be avoided for that for example polymer layers in the fluid chamber and nozzle may start to melt. Further, if water was being ejected, at temperatures around 95° C.-100° C. drops would start to shoot out of the fluid chamber in an uncontrolled manner. So, in general, the liquid in the assembly can be heated to a similar temperature as the die, wherein an ejecting a ‘warm’ drop of fluid can also create a warmer vapor for inhalation. FIG. 2B shows a schematic view of a MEMS layout in a substrate according to an embodiment of the present invention. There is specifically shown an exemplary position of the TSR 401 on a MEMS device 40. By means of the TSR solution, there can be obtained a reduction on the particle/droplet size. Especially, when the air temperature is enough there can be vaporized small droplets in the size range of <8 μm. By use of the liquid jet device technology there can be generated small droplets, and, in turn, the benefit would be obtained of smaller droplet sizes due to the vaporization of those.

Further, this purpose and functionality may also be combined with heating resistor 42 arranged in a vicinity of said fluid chamber 41 and configured to heat a first amount of the liquid 46 to at least a vaporization temperature, so that a vapour bubble 47 expels a drop 45 of the liquid 46 through the ejection nozzle 44. For example, the resistor 42 may be driven to heat the fluid chamber 41 and/or the supply channel 43 to a temperature below a threshold that would result in forming the bubble 47 and, consequently, in expelling the drop/droplet 45. Yet still, the relevant part of the liquid 46 could be at a predetermined temperature prior to ejection. In a way, this can be named as pulse warming by providing a pulse that is for example half the time as a firing pulse, wherein the heater warms up significantly but not enough to eject droplets. Through thermal conduction the liquid in the chamber can thus be increased. When this liquid is ejected also the aerosol or vapor temperature is increased for inhalation.

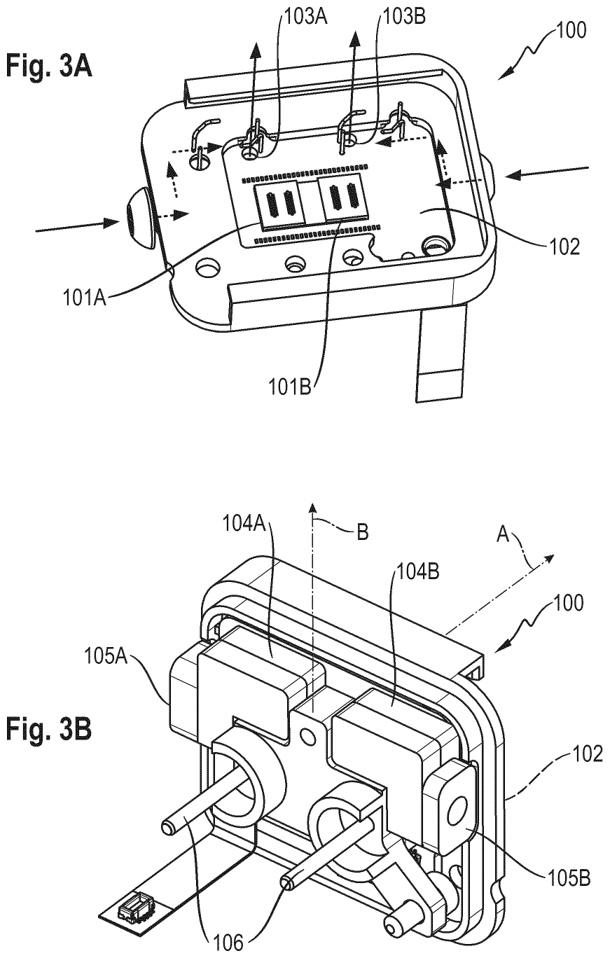

FIGS. 3A and 3B show schematic views of a vapour generator assembly 100 of an inhalation device according to respective embodiments of the present invention. In FIG. 3A there is shown the vapour generator assembly 100 from a front view from a side of the mixing chamber. As can be seen, there are two liquid jet device 101A, 101B in the form of micro-electromechanical systems (MEMSs) on a circuit board 102, wherein the inlet air is guided through opening 103A, 103B of said printed circuit board 102. The mixing chamber may be formed by placing an airtight cover on the circuit board which then acts as a rear limitation of the chamber, in which the air penetrating through the inlet opening 103A, 103B can mix with the drops generated by the liquid jet devices 101A, 101B. The cover may provide an outlet toward a mouthpiece or toward a user for inhalation.

In such a configuration the control, for example implemented by the already mentioned control unit 17, may comprise controlling a temperature of the drops. For such a purpose, the inhalation device comprises a respective heating element arranged to pre-heat said liquid to a predetermined temperature prior to ejection through said ejection nozzle. For example, the heating element can be a resistor embedded in a substrate of said MEMS so as to pre-heat to the predetermined temperature at least a part of the liquid present in the substrate. In principle, any resistor can be used as both power delivery to the device and measurement of resistance on the device. A resistor can thus for example increase the fluid temperature before ejection in a piezo-type jet device.

In FIG. 3B there is shown the vapour generator assembly 100 from a rear view from a side underneath the rear limitation of of the mixing chamber (e.g. in the form of a printed circuit board). In this embodiment there may be provided at least two heating elements 104A, 104B, one each for the inlet orifices 105A, 105B. The heating elements 104A, 104B may be configured and arranged symmetrically relative to an axis A or B in the inhalation device. Preferably, a heating element comprises a curved air flow path or even a meandering air flow path, so as to ensure sufficient and reliable heat exchange. In some embodiments the heating elements may comprise a ceramic heater housing. In other words, the airpath can be heated up before reaching the drop on demand generator. Air enters into the device through an orifice from which the air passes over one or more heating coils to provide convection heating of the air. The warm air enters the mouthpiece chamber where it is combined with the drops, preheated or at room/ambient temperature. This vapor mixture enters the mouth above ambient temperature.

In addition to the above, fluid intake channels 106, for example in the form of thin tubes or hollow needles, may extend downward from underneath the liquid jet devices mounted on the printed circuit board. These channels 106 may be in fluid communication with one or more reservoirs for storing one or more liquids as a base substance for vapor generation. In this way, more than one base substance can be employed to generate an adjustable and controllable mixture of several agents, flavours, etc.

Although detailed embodiments have been described, these only serve to provide a better understanding of the invention defined by the independent claims and are not to be seen as limiting.

1. An inhalation device with at least one liquid jet device for producing drops of a liquid on demand,
said liquid jet device comprising a fluid chamber, an ejection nozzle and a supply channel embedded in a substrate,
the inhalation device further comprising at least one heating element arranged to pre-heat said liquid to a predetermined temperature prior to ejection through said ejection nozzle.

2. The inhalation device according to claim 1, wherein the liquid jet device is in the form of a micro-electromechanical system (“MEMS”).

3. The inhalation device according to claim 2, wherein the heating element is a resistor embedded in a substrate of said MEMS so as to pre-heat to the predetermined temperature at least a part of the liquid present in cavities formed in the substrate.

4. The inhalation device according to claim 2, wherein the resistor is a temperature sense resistor (“TSR”) embedded in the substrate.

5. The inhalation device according to claim 3, wherein the resistor is a heating resistor arranged in a vicinity of said fluid chamber and configured to heat an amount of the liquid in said fluid chamber to at least a vaporization temperature, so that a vapour bubble expels a drop of the liquid through the ejection nozzle.

6. The inhalation device according to claim 5, further comprising a control unit configured to drive said resistor so that said amount of the liquid in the firing chamber is pre-heated to said predetermined temperature prior to heating said amount of the liquid to at least the vaporization temperature.

7. The inhalation device according to claim 6, wherein said control unit is configured to apply a voltage pulse with variable width to said resistor, and wherein a pre-heating pulse is shorter than a vaporization pulse.

8. The inhalation device according to claim 7, wherein said pre-heating pulse is approximately half as long as said vaporization pulse.

9. The inhalation device according to claim 1, further comprising an air conduit and a mouthpiece chamber in which air from said air conduit is mixed with the generated liquid drops.

10. The inhalation device according to claim 9, wherein the air conduit comprises a further heating element arranged to pre-heat the air guided by the air conduit into the mixing chamber.

11. The inhalation device according to claim 1, further comprising a reservoir configured to store an amount of said liquid and a reservoir heating element arranged to heat the liquid in said reservoir to a predetermined liquid reservoir temperature.

12. The inhalation device according to claim 1, comprising at least one second liquid jet device for producing drops of a liquid on demand, said at least one second liquid jet device comprising a fluid chamber, an ejection nozzle and a supply channel embedded in a further substrate.

13. The inhalation device according to claim 12, comprising at least one second heating element arranged to pre-heat a liquid to a further predetermined temperature prior to ejection through the ejection nozzle in the further substrate.

14. The inhalation device according to claim 13, wherein the at least one liquid jet device and said at least one second liquid jet device are configured for producing drops from respective different liquids.

15. The inhalation device according to claim 13, wherein the at least one liquid jet device and said at least one second liquid jet device are configured for producing drops from the same liquid.

16. The inhalation device according to claim 12, wherein the at least one liquid jet device and said at least one second liquid jet device are in the form of a micro-electromechanical system (“MEMS”) and are formed in the same substrate.

17. The inhalation device according to claim 1, wherein the at least one liquid jet device is mounted on a printed circuit board, inlet air is guided through an opening of said printed circuit board, and a mixing chamber is arranged atop the printed circuit board so that the at least one liquid jet device produce drops into said mixing chamber.
